(19) United States
(12) Patent Application Publication
(10) Pub. No.: US 2020/0179098 A1
(43) Pub. Date: Jun. 11, 2020

(54) DOUBLE LEAD INTERFERENCE SCREW

(71) Applicants: Robert Marx, New York, NY (US); William Ragheb, Miami, FL (US); Smith & Nephew. Inc., Memphis, TN (US); Smith & Nephew Orthopaedics AG, Baar (CH); Smith & Nephew Pte. Limited, Singapore (SG)

(72) Inventors: Jennifer Ng, Seattle, WA (US); Robert Marx, New York, NY (US); William Ragheb, Miami, FL (US); Jared Harlow, Palmyra, PA (US); Jeffrey Wyman, Naples, FL (US); Stephen Santangelo, Sturbridge, MA (US)

(21) Appl. No.: 16/210,269

(22) Filed: Dec. 5, 2018

(51) Int. Cl. A61F 2/08

(52) U.S. Cl. CPC A61F 2/0811; A61F 2002/0858; A61F 2002/0882

(57) ABSTRACT

A double lead interference screw useful for securing a soft tissue graft inside a bone tunnel of a bone mass is disclosed. The interference screw has continuous independent double threads having uniform pitch, helix angle, and crest height throughout the threaded portions of the screw; a bulbous head; a threaded proximal section with a cylindrical root, a threaded distal section with a tapered root, and a threaded tapered distal tip. Methods for soft tissue repair using the double lead interference screw are also disclosed.

### FIELD OF THE INVENTION

The present invention generally relates to the field of medical fixation devices used for soft tissue repair, and more particularly relates to interference screws used generally for fixation of soft tissue grafts to a bone mass.

### BACKGROUND OF THE INVENTION

Anterior Cruciate Ligament (ACL) reconstruction is one of the most common arthroscopic knee procedures performed on patients. A variety of grafts can be used to reconstruct the ACL, however, a soft tissue graft such as a hamstring or quadriceps tendon is largely used. There is a need to fixate the grafts in the bone tunnels that are drilled during the procedure. Many techniques for soft tissue repair are known in the art. For example, common methods for fixation include: suspensory fixation, use of spike plates, and use of interference screws. The fixation methods vary depending on whether they are used in soft tissue grafts or bone-tendon-bone grafts. There are many fixation devices on the market including interference screws. Interference screws can be made a variety of materials, including metal, polyetheretherketone (PEEK), or a bioabsorbable material such as poly-L lactic acid (PLLA). For fixation of soft tissue grafts using interference screws, an interference screw is inserted into a pre-drilled bone tunnel through which a soft tissue graft passes to secure the graft to the bone by compressing the graft against the surface of the tunnel with a sufficient fixation strength to hold the graft in place. However, some interference screws are difficult to insert because of their design, requiring excessive force for insertion, which can cause graft cutting during insertion, especially with metal interference screws. Thus, it would be advantageous for an interference screw to have a design which allows for easy insertion and still have sufficient fixation strength for soft tissue grafts.

### SUMMARY OF THE INVENTION

The present invention provides a solution to the aforementioned limitations and deficiencies in the art relating to interference screws used for securing soft tissue grafts to a bone mass, such as in ACL reconstruction. The solution is premised on the interference screw of the invention, which has a novel design that not only requires less force for insertion into bone tunnels thereby increasing ease of insertion, but also provides greater fixation strength as compared to other interference screws currently commercially available on the market. The interference screw is useful for securing soft tissue grafts to a bone mass and can be used for any type of soft tissue repair requiring the use of an interference screw, including ACL reconstruction. The interference screw can also be used to secure bone-tendon-bone grafts to a bone mass.

An embodiment of the invention is a double lead interference screw having a proximal end and a distal end; a bulbous head positioned at the proximal end having rounded edges and a flat top profile; continuous double threads (double lead) having uniform pitch, helix angle, and crest height, extending from the distal end to the bulbous head; a threaded tapered distal tip positioned at the distal end; a threaded proximal section having a cylindrical (constant diameter) root positioned distal to the bulbous head; and a threaded distal section having a tapered root positioned between the proximal section and the tapered distal tip, wherein the tapered root of the distal section gradually reduces in diameter from the proximal section to the distal tip, and wherein the crest diameter of the threads of the distal section narrows proportionally to the taper of the tapered root of the distal section maintaining a uniform crest height.

In various embodiments, the interference screw can be made of any biocompatible material suitable for use in an interference screw including but not limited to a metal material such as titanium or stainless steel, a polymer such as polyether ether ketone (PEEK), or a bioabsorbable material such as poly-L-lactic acid (PLLA), hydroxyapatite (HA), or PLLA-HA. In some embodiments, the interference screw is made of metal. In some embodiments, the metal is titanium. In other embodiments, the metal is stainless steel.

In some embodiments, the interference screw is cannulated having a bore extending longitudinally through the center (axis) of the screw from the proximal end to the distal end. In some embodiments, the bore is cylindrical. In various embodiments, the bore has varying diameters and/or shapes.

In some embodiments, the bulbous head of the interference screw has a recess configured to receive a drive shaft or screw driver. In some embodiments, the recess is a hexagonal socket.

In some embodiments, the threaded proximal section has a crest diameter of 9 mm, and the crest diameter of the threaded tapered distal section narrows (steps) from 9 mm to 7 mm (2 mm step).

In some embodiments, the overall length of the interference screw is 30 mm, the length of the threaded proximal section is 15 mm, and the length of the threaded distal section is 5 mm. In some embodiments, the overall length of the screw is 25 mm, the length of the threaded proximal section is 10 mm, and the length of the threaded distal section is 5 mm. In some embodiments, the overall length of the screw is 20 mm, the length of the threaded proximal section is 5 mm, and the length of the threaded distal section is 5 mm. In some embodiments, the length of the bulbous head is 5 mm and the length of the threaded distal tip is 5 mm. In some embodiments, the length of the threaded distal section (not including the threaded distal tip) is equal to or less than the length of the threaded proximal section.

Other embodiments and aspects of the invention include methods of soft tissue repair using the interference screw of the invention. An embodiment of the invention is a method of attaching a soft tissue graft to a bone mass for repair/replacement of damaged soft tissue, the method comprising: (a) drilling a bone tunnel of a constant diameter into the bone mass to which the soft tissue graft is to be attached, (b) placing the soft tissue graft partially or completely through the bone tunnel against a portion of the interior wall of the bone tunnel, (c) positioning the distal end of the double lead interference screw of the invention at the opening of the bone tunnel, and (d) rotating the interference screw with sufficient torsional force while applying sufficient axial force to advance the screw into the bone tunnel until the proximal end of the screw is flush or substantially flush with the outer surface of the bone mass.

In some embodiments, the interference screw is cannulated, and the method further comprises positioning a guide wire through the cannulated interference screw and into the bone tunnel to guide the screw during insertion. In some embodiments, the bulbous head of the interference screw has a recess in the head configured to receive a drive shaft, and the method further comprises inserting the drive shaft of a screw driver into the recess in the head, wherein the torsional and axial forces are applied using the screw driver. In some embodiments, the diameter of the bone tunnel is about 1 mm greater than the crest diameter of the threaded proximal section of the interference screw. In some embodiments, the damaged soft tissue is an anterior cruciate ligament (ACL). In some embodiments, the soft tissue graft is a hamstring tendon or a quadriceps tendon. In some embodiments, the bone mass is a femur bone and/or a tibia bone.

Other objects, features and advantages of the present invention will become apparent from the following detailed description. It should be understood, however, that the detailed description, the illustrations, and the specific examples, while indicating specific embodiments of the invention, are given by way of illustration only, since various changes and modifications within the spirit and scope of the invention will become apparent to those skilled in the art from this detailed description.

The feature or features of one embodiment may be applied to other embodiments, even though not described or illustrated, unless expressly prohibited by this disclosure or the nature of the embodiments. Details associated with the embodiments described above and others are presented below.

### BRIEF DESCRIPTION OF THE DRAWINGS

FIG. 1 is a side view of the interference screw of the invention.

FIG. 2 is a top view of the interference screw of FIG. 1.

FIG. 3 is a bottom view of the interference screw of FIG. 1.

FIG. 4 is a diagram representing the crest diameter of the threaded proximal section and threaded distal section of the interference screw (threads not shown).

FIG. 5 is a side view of a 9×20 mm interference screw, a 9×25 mm interference screw, and a 9×30 mm interference screw.

FIG. 6 is a photo of various interferences screws used in comparative studies.

FIG. 7 is a graph comparing the fixation strengths of various interference screws.

FIG. 8 is a graph comparing the average insertion load of various interference screws used in an ease of insertion study.

FIG. 9 is a graph comparing the average torque of various interference screws used in an ease of insertion study.

### DESCRIPTION OF THE ILLUSTRATED EMBODIMENTS

The term “root” also known as “core” or the “root of the thread” as used herein means the innermost surface joining the two sides (flanks) of a thread.

The term “crest” also known as the “crest of the thread” as used herein means the outermost surface joining the two sides (flanks) of a thread.

The term “crest height” also known as the “depth of the thread” as used herein means the distance between the crest and the root of a thread.

The term “crest diameter” also known as “outer diameter” as used herein means the diameter at the crest of the thread measured at right angle to the axis.

The term “helix angle” as used herein means the angle formed by a point on the side and the plane perpendicular to the axis of the screw thread.

The term “pitch” as used herein means the distance from the crest of one thread to the crest on an adjacent thread, measured parallel to the axis of the screw.

The term “double threads” also known as “double helix,” “double-start thread,” or “two-start thread” as used herein with respect to an interference screw means two independent continuous threads running parallel to each other along the length of the threaded portions of the screw.

The term “lead” as used herein means the axial distance that the screw advances in one complete turn (360°).

The term “double lead” as used herein means the lead of a screw equal to twice the pitch, indicative of a screw having double threads (two-start thread).

The term “cannulated” as used herein with respect to an interference screw means an interference screw having a bore extending longitudinally through the center (axis) of the screw from the proximal end to the distal end. The bore can be cylindrical or can have varying diameters and/or shapes.

The use of the word “a” or “an” when used in conjunction with the terms “comprising”, “having”, “including”, or “containing” (or any variations of these words) may mean “one,” but it is also consistent with the meaning of “one or more,” “at least one,” and “one or more than one,” unless this disclosure explicitly requires otherwise.

The terms “about” or “approximately” are defined as being close to as understood by one of ordinary skill in the art, and in one non-limiting embodiment the terms are defined to be within 10%, preferably within 5%, more preferably within 1%, and most preferably within 0.5%.

The terms “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive and open-ended, and do not exclude additional, unrecited components, features, or elements. These terms can be used in any of the claims. The terms consisting essentially of (and any form thereof, such as “consist essentially of” and “consists essentially of”), and “consisting” (and any form of consisting, such as “consist” and “consists”) can be can be substituted for any of the open-ended terms recited above and can be used in any of the claims in order to change the scope of a given claim from what it would otherwise be using the open-ended term.

The interference screw of the invention has a novel design that not only requires less force for insertion into bone tunnels thereby increasing ease of insertion, but also provides for greater fixation strength as compared to other interference screws currently available on the market. The interference screw is useful for securing soft tissue grafts to a bone mass and can be used for any type of soft tissue repair requiring the use of an interference screw, including ACL reconstruction. The interference screw can also be used to secure bone-tendon-bone grafts to a bone mass.

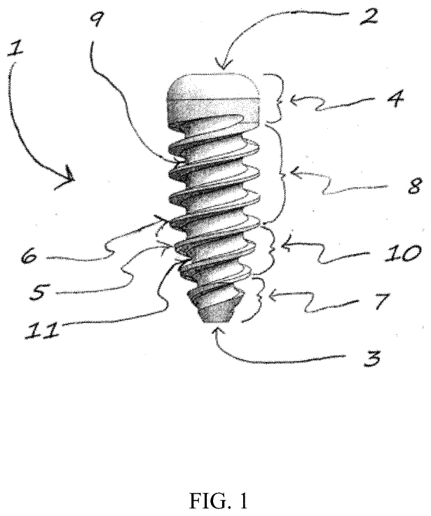

Now turning to FIG. 1, shown is a side view of an embodiment of the double lead interference screw 1 having a proximal end 2 and a distal end 3; a bulbous head 4 positioned at the proximal end having rounded edges and a flat top profile; continuous double threads 5, 6 having uniform pitch, helix angle, and crest height, extending from the distal end to the bulbous head; a threaded tapered distal tip 7 positioned at the distal end; a threaded proximal section 8 having a cylindrical (constant diameter) root 9 positioned distal to the bulbous head; and a threaded distal section 10 having a tapered root 11 positioned between the proximal section and the tapered distal tip, wherein the tapered root of the distal section gradually reduces in diameter from the proximal section to the distal tip, and wherein the crest diameter of the threads of the distal section narrows proportionally to the taper of the tapered root of the distal section thus maintaining a uniform crest height. The threaded proximal section has a cylindrical (constant diameter) root and thus, has a constant crest diameter. The two tapered sections of the interference screw, i.e., the threaded tapered distal section and the threaded tapered distal tip, facilitate screw insertion into a bone tunnel and provide sufficient fixation strength for soft tissue grafts once fully inserted. This double tapered design of the screw can also prevent the cutting of soft tissue grafts during insertion into bone tunnels. The interference screw's continuous double threads (two-start thread) provide the screw with a double lead, which enables faster bite to the cortical bone and less rotations to advance the screw compared to a screw with a single thread (one-start thread). The double threads are two independent continuous threads having uniform pitch, helix angle, and crest height running parallel to each other along the length of the threaded portions of the screw. In various embodiments, the double threads of the interference screw can be of any thread types. Non-limiting examples of thread types include V-shaped threads, square threads, buttress threads, and reverse buttress threads. In some embodiments, the double threads are V-shaped threads. The screw can have normal (right-hand) or reverse (left-hand) threads. The bulbous head of the interference screw is non-threaded, has rounded edges to prevent the screw from fraying or cutting the soft tissue graft when the patient moves the repaired joint, and has a flat top profile (perpendicular to the axis of the screw) at the proximal end (top of screw) which allows the screw to be flush or substantially flush with the cortical bone when fully inserted further preventing damage to the graft during movement of the repaired joint.

In various embodiments, the interference screw can be made of any biocompatible material including, but not limited to, a metal material such as titanium or stainless steel, a polymer such as polyetheretherketone (PEEK), or bioabsorbable materials such as poly-L-lactic acid (PLLA), hydroxyapatite (HA), or PLLA-HA. Other non-limiting examples of metal materials include steel alloys, cobalt chrome alloys, zirconium, oxidized zirconium, tantalum, titanium alloys, and titanium-nickel alloys. Other non-limiting examples of polymer materials include polyamide base resins, polyethylene, ultra high molecular weight (UHMW) polyethylene, low density polyethylene, polymethylmethacrylate (PMMA), polyetherketoneketone (PEKK), and polyurethane, any of which may be reinforced.

In some embodiments, the interference screw is cannulated having a bore extending longitudinally through the center (axis) of the screw from the proximal end to the distal end. The bore can be cylindrical or can have varying diameters and/or shapes. A cannulated screw can be useful for receiving guide wires to direct the screw to a desired mounting position.

In some embodiments, the top of the bulbous head of the interference screw has a recess configured to receive a drive shaft or screw driver. Non-limiting examples of a recess suitable for receiving a drive shaft or screw driver include a hexagonal socket, a star socket (TORX), a Philips, a square socket, and a double square socket. In some embodiments, the recess is a hexagonal socket.

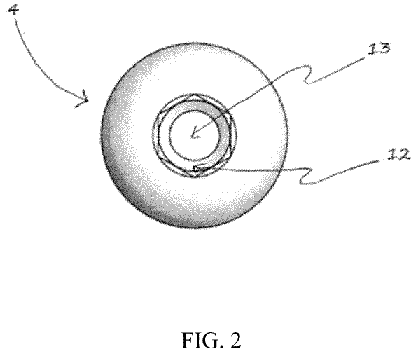

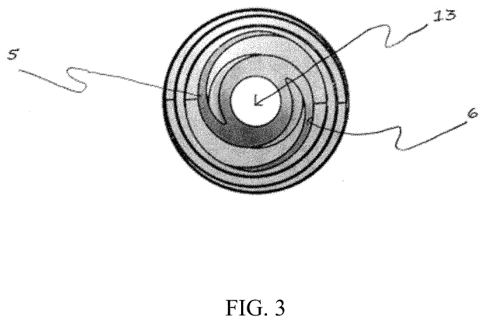

In some embodiments, the interference screw is cannulated and the bulbous head of the screw has a recess configured to receive a drive shaft or screw driver. Now turning to FIG. 2, shown is a top view (proximal) of a cannulated embodiment of interference screw of FIG. 1 showing a hexagonal socket recess 12 in the bulbous head 4 and the proximal end of the bore 13. Now turning to FIG. 3, shown is a bottom view (distal) of a cannulated embodiment of interference screw of FIG. 1 showing the double threads 5 and 6 and the distal end of the bore 13.

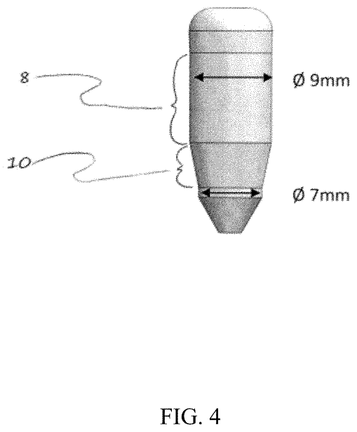

In various embodiments, the constant crest diameter of the threaded proximal section can be any diameter suitable for insertion into a given bone tunnel, and the crest diameter of the threaded tapered distal section can narrow from the crest diameter of the threaded proximal section down to any diameter less than the crest diameter of the threaded proximal section. In some embodiments, the threaded proximal section has a crest diameter of 9 mm, and the crest diameter of the threaded distal section narrows (steps) from 9 mm to 7 mm (2 mm step). Now turning to FIG. 4, shown is a diagram representing the crest diameter of the threaded proximal section 8 (threads not shown) and the threaded distal section 10 (threads not shown) of an interference screw, illustrating an embodiment of a screw having a constant crest diameter of the threaded proximal section of 9 mm and a tapered crest diameter of the threaded distal section narrowing from 9 mm to 7 mm (2 mm step).

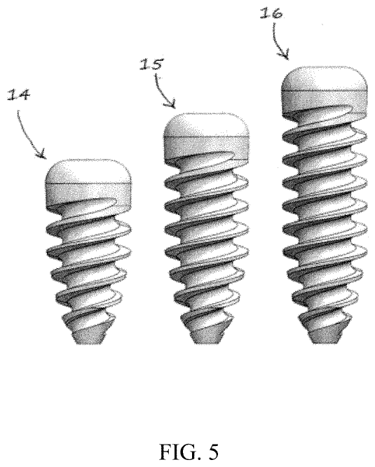

In various embodiments, the overall length of the interference screw and the lengths of the various portions of the screw, i.e., the bulbous head, the threaded proximal section, the threaded tapered distal section, and the threaded tapered distal tip, can be of any length suitable for fixation of a tissue graft in a given bone tunnel. In some embodiments, the overall length of the interference screw is 30 mm, the length of the threaded proximal section is 15 mm, and the length of the threaded distal section is 5 mm. In some embodiments, the overall length of the screw is 25 mm, the length of the threaded proximal section is 10 mm, and the length of the threaded distal section is 5 mm. In some embodiments, the overall length of the screw is 20 mm, the length of the threaded proximal section is 5 mm, and the length of the threaded distal section is 5 mm. In some embodiments, the length of the bulbous head is 5 mm and the length of the threaded distal tip is 5 mm. In some embodiments, the length of the threaded tapered distal section (not including the threaded distal tip) is equal to or less than the length of the threaded proximal section. Now turning to FIG. 5, shown is a side view of three embodiments of the interference screw: interference screw 14, interference screw 15, and interference screw 16 with dimensions shown in Table 1 below.

TABLE 1

| Interference Screw | | | |
|---|---|---|---|
| number as shown in FIG. 5: | 14 | 15 | 16 |
| Overall length (mm) | 20 | 25 | 30 |
| Constant Crest | 9 | 9 | 9 |
| Diameter of Threaded | | | |
| Proximal Section (mm) | | | |
| Narrowest Crest | 7 | 7 | 7 |
| Diameter of Threaded | | | |
| Distal Section (mm) | | | |
| Length of Bulbous Head | 5 | 5 | 5 |
| (mm) | | | |
| Length of Threaded | 5 | 10 | 15 |
| Proximal Section (mm) | | | |
| Length of Threaded | 5 | 5 | 5 |
| Distal Section (mm) | | | |
| Length of Threaded | 5 | 5 | 5 |
| Distal Tip (mm) | | | |

Other embodiments and aspects of the invention include methods of soft tissue repair or replacement using the dual lead interference screw of the invention described throughout this disclosure. The interference screw can be used for any type of soft tissue repair or replacement requiring the use of an interference screw including, but not limited to, ACL reconstruction. In soft tissue repair/replacement surgery, the interference screw is used to secure a soft tissue graft to a pre-drilled bone tunnel in a bone mass.

An embodiment of the invention is a method of attaching a soft tissue graft to a bone mass for repair/replacement of damaged soft tissue, the method comprising: (a) drilling a bone tunnel of a constant diameter into the bone mass to which the soft tissue graft is to be attached, (b) placing the soft tissue graft partially or completely through the bone tunnel against a portion of the interior wall of the bone tunnel, (c) positioning the distal end of the double lead interference screw of the invention at the opening of the bone tunnel, and (d) rotating the interference screw with sufficient torsional force while applying sufficient axial force to advance the screw into the bone tunnel until the proximal end of the screw is flush or substantially flush with the outer surface of the bone mass. The term “substantially flush” as used herein means that all or a portion of the top of the screw (proximal end) can be slightly above or below the outer surface of the bone mass, i.e., up to 1 mm, or up to 2 mm, or up to 3 mm, or up to 4 mm, or up to 5 mm above or below the outer surface of the bone mass, as long as the screw does not damage the tissue graft during movement of the repaired joint. Because of the novel design of the interference screw of the invention, the insertion of the screw into the bone tunnel is easier because of less forces needed to advance the screw compared to other commercially available interference screws currently on the market and provides sufficient fixation strength for soft tissue grafts once fully inserted. Another benefit of the design of the interference screw of the invention is the prevention or minimization of soft tissue graft cutting during insertion of the screw into bone tunnels.

The methods can further include the use of guide wires with cannulated interference screws to help guide the screw into the proper position. In some embodiments, the interference screw is cannulated, and the method further comprises positioning a guide wire through the cannulated interference screw and into the bone tunnel to guide the screw during insertion. A screw driver with a drive shaft can be used with interference screws that have a recess in the head of the screw configured to receive a drive shaft. In some embodiments, the bulbous head of the interference screw has a recess in the head configured to receive a drive shaft, and the method further comprises inserting the drive shaft of a screw driver into the recess in the head, wherein the torsional and axial forces are applied using the screw driver. Generally, the diameter of the bone tunnel can be about 1 mm greater than the largest outer diameter of the interference screw, which would be the crest diameter of the threaded proximal section of the interference screw of the invention. In some embodiments, the diameter of the bone tunnel is about 1 mm greater than the crest diameter of the threaded proximal section of the interference screw.

The interference screw of the invention can be used for any type of soft tissue repair or replacement requiring the use of an interference screw, including but not limited to ACL reconstruction. In soft tissue repair/replacement, the interference screw is used to secure a soft tissue graft to a bone mass. The soft tissue graft can be any suitable soft tissue graft type including but not limited to autografts, allografts, and synthetic grafts. Non-limiting examples of an autograft include the hamstring tendon and the quadriceps tendon. Non-limiting examples of an allograft from a cadaver include the patellar ligament, the tibialis anterior tendon, and the Achilles tendon. In some embodiments, the soft tissue graft is a hamstring tendon or a quadriceps tendon. The damaged soft tissue can be any soft tissue in need of repair or replacement, and generally is a ligament or tendon. In some embodiments, the damaged soft tissue is an anterior cruciate ligament (ACL). In ACL reconstruction surgery, the soft tissue graft is generally attached to the femur bone and/or the tibia bone. In some embodiments, the bone mass is a femur bone and/or a tibia bone.

### EXAMPLES

### Example 1

### Fixation Strength Comparative Study

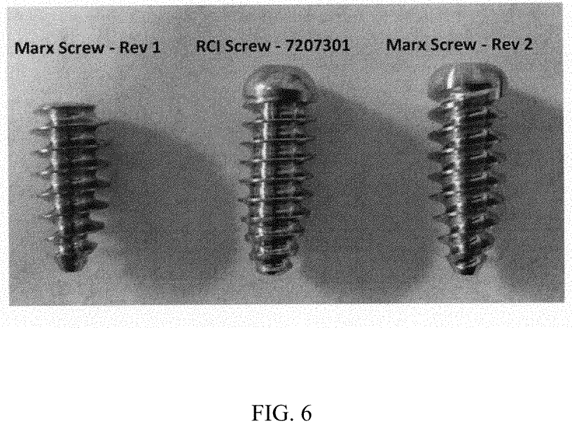

A comparative study using a synthetic graft fixation model was conducted to determine the soft tissue fixation capabilities between several interference screw types currently commercially available and two prototype interference screws one of which is an embodiment of the interference screw of the invention. Testing was done on four commercially available interference screw types identified as 7×20 RCI (titanium), 9×20 RCI (titanium), 7×20 RCI-HA and 9×20 RCI-HA available from Smith & Nephew, one prototype interference screw type identified as “Marx Screw-Rev 1,” and another prototype interference screw type, identified as “Marx Screw-Rev 2,” which is an embodiment of the interference screw of the invention. The Marx Screw-Rev 2 is a double lead interference screw made of titanium that has double threads, a bulbous head, and has the same dimensions as stated for interference screw 15 in Table 1 above. The Marx Screw-Rev 2 is pictured in FIG. 6.

Simulated soft tissue was made of 11 in long braided nylon rope sections with a 125 lb break strength obtained from Bridgeline Rope Inc. (REF 4022 SP). Simulated bone blocks were made of polyurethane having a density of 10 pcf obtained from Pacific Research Laboratories, Tacoma WA (REF 1522-01). Tunnels were drilled perpendicular to the bone block surface completely through the bone block. The diameter of the tunnel was based on the largest outer diameter of the test screw plus 1 mm, e.g., for a 9 mm outer diameter screw, the tunnel diameter was 10 mm. For each test screw trial, two 11 in long braided nylon rope sections were wetted and the core was removed. The two ropes were doubled over and were slid through the tunnel using a suture. The loops of the two doubled over ropes on one side of the block were placed around a large pin gauge on a fixture stand. The block was placed on the stand between the large pin gauge and two medium pin gauges. Each of the four strands of the ropes on the other side of the block were placed in each quadrant of the tunnel and then held in tension using Extreme clothespins. The test screw was then inserted in the tunnel between the four strands of the ropes until the top of the screw was flush with the bone block. The ropes were allowed to dry for at least 12 hours.

The study was conducted using an MTS Insight® 5 material testing system available from MTS Systems Corporation, Eden Prairie, Minn. with a 5 kN load cell. The test article was placed within a slotted test stand such that the rope/screw construct would be pulled parallel to the bone tunnel and the loops of the ropes were placed over the hook of the testing system. The testing system was then activated which pulled the rope construct from the bone block opposite the direction of the screw insertion and the peak load (fixation strength) in Newtons (N) was recorded. Data was collected at a frequency of 50 Hz.

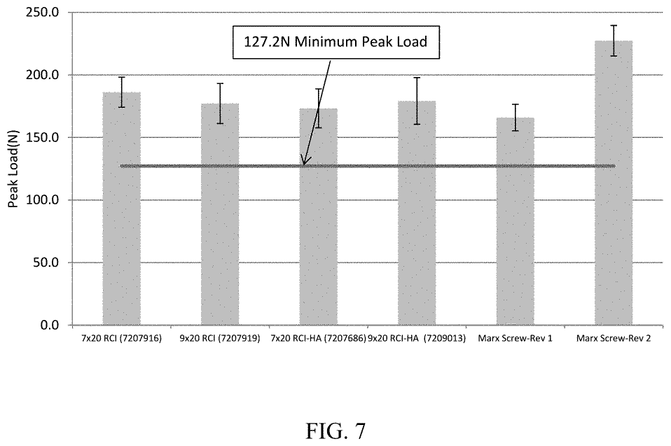

Fifteen screws of each screw type were tested 2 times each for a total of 30 trials for each screw type. The minimum pass/fail criteria for acceptable fixation strength (minimum peak load) is 127.2 N. The results of the study are shown in Table 2 below and in FIG. 7.

TABLE 2

| | Screw Type | | | | | |
|---|---|---|---|---|---|---|
| | | | | | Marx | Marx |
| | 7 × 20 | 9 × 20 | 7 × 20 | 9 × 20 | Screw- | Screw- |
| | RCI | RCI | RCI-HA | RCI-HA | Rev 1 | Rev 2 |
| Mean Fixation | 186.1 | 177.2 | 173.3 | 179.2 | 165.9 | 227.3 |
| Strength (N) | | | | | | |
| Std. Dev (N) | 12.0 | 16.0 | 15.5 | 18.6 | 10.6 | 12.2 |

As can be seen in the results, the interference screw of the invention (Marx Screw-Rev 2) had the greatest fixation strength of all the screws tested having an increase of at least 22% in average fixation strength and an increase of 28% in comparison to the commercially available interference screw 9×20 RCI.

### Example 2

### Ease of Insertion Comparative Study

A comparative study was conducted to determine the ease of insertion of three interference screw types: a commercially available interference screw identified as RCI Screw -7207301; the prototype screw identified as “Marx Screw-Rev 1,” from Example 1; and another prototype interference screw, identified as “Marx Screw-Rev 2” from Example 1 which is an embodiment of an interference screw of the invention. The RCI Screw -7207301 is a 9×25 mm interference screw made of titanium available from Smith & Nephew. The “Marx Screw-Rev 2” is a double lead interference screw made of titanium that has double threads, a bulbous head, and has the same dimensions as stated for interference screw 15 in Table 1 above. The test screws are pictured in FIG. 6.

Simulated soft tissue was made of 11 in long braided nylon rope sections with a 125 lb break strength obtained from Bridgeline Rope Inc. (REF 4022 SP). Simulated bone blocks were made of polyurethane having a density of 10pcf obtained from Pacific Research Laboratories, Tacoma Wash. (REF 1522-01). Tunnels were drilled perpendicular to the bone block surface completely through the bone block. The diameter of the tunnel was based on the largest outer diameter of the test screw plus 1 mm, e.g., for a 9 mm outer diameter screw, the tunnel diameter was 10 mm. For each test screw trial, two 11 in long braided nylon rope sections were wetted and the core was removed. The two ropes were doubled over and were slid through the tunnel using a suture. The loops of the two doubled over ropes on one side of the block were placed around a large pin gauge on a fixture stand. The block was placed on the stand between the large pin gauge and two medium pin gauges. Each of the four strands of the ropes on the other side of the block were placed in each quadrant of the tunnel and then held in tension using Extreme clothespins.

The study was conducted using an ADMET eXpert™ Model 8602 Axial-Torsion Testing Machine available from ADMET Inc., Norwood, Mass. The ADMET testing machine was programed with ASTM standard test method “F543-A4.” Adaptations were made to the program to simulate soft tissue insertion as opposed to bone graft insertion. The bone block with tensioned ropes was loaded into the vice of the ADMET testing machine for screw insertion. A guide wire was placed in the tunnel. The test screw was attached onto the driver and positioned on the head of the testing machine. The head was lowered until the screw was about to touch the ropes in the tunnel. The program was started and the testing machine began to insert the screw. The program calls for the screw to be advanced at a constant rotation and with increasing axial force. This continues until the transducer senses an increase in torsional forces. Once sensing the threads are engaged, the testing machine will stop the increasing axial force and allow the screw to continue advancing itself through rotation. The position, torque in Newton meters (Nm) and load in Newtons (N) were collected at a frequency of 10 Hz throughout the insertion of the screw.

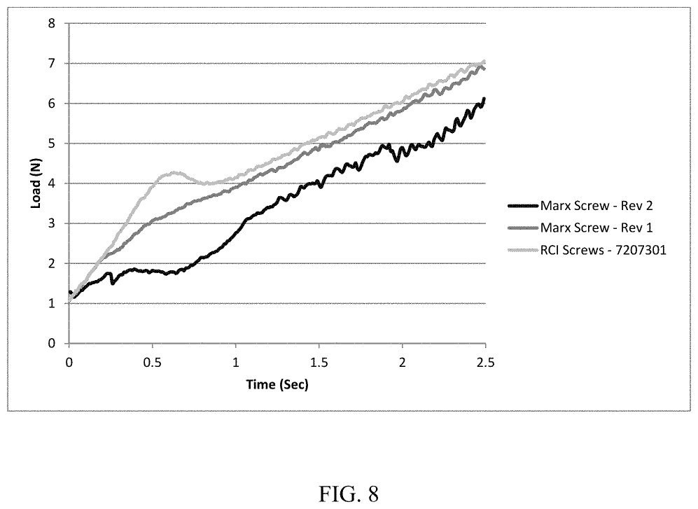

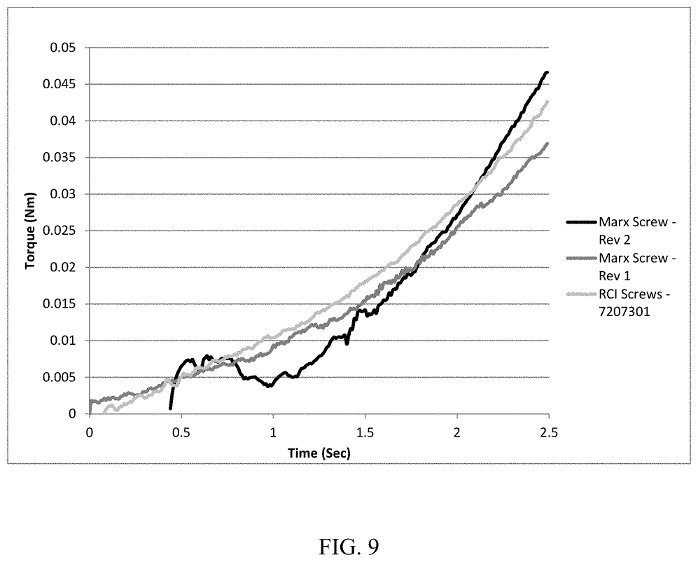

The test was conducted on 15 screws of each screw type tested. The data collected from the study includes both the axial load applied shown in FIG. 8 as well as the torsional force applied shown in FIG. 9. As can be seen in FIG. 8, the interference screw of the invention (Marx Screw-Rev 2) required an average of 28% less force to insert than did the commercially available screw RCI-7207301. As can be seen in FIG. 9, the interference screw of the invention (Marx Screw-Rev 2) required an average of 16% less torque for insertion than did the commercially available screw RCI-7207301.

Terms such as distal, proximal, bottom, top, side, away, near, over, and the like have been used relatively herein. However, such terms are not limited to specific coordinate orientations, distances, or sizes, but are used to describe relative positions referencing particular embodiments. Such terms are not generally limiting to the scope of the claims made herein. Any embodiment or feature of any section, portion, or any other component shown or particularly described in relation to various embodiments of similar sections, portions, or components herein may be interchangeably applied to any other similar embodiment or feature shown or described herein.

While embodiments of the invention have been illustrated and described in detail in the disclosure, the disclosure is to be considered as illustrative and not restrictive in character. All changes and modifications that come within the spirit of the invention are to be considered within the scope of the disclosure.

1. A double lead interference screw having a proximal end and a distal end comprising:
(a) a bulbous head positioned at the proximal end having rounded edges and a flat top profile;
(b) continuous double threads (double lead) having uniform pitch, helix angle, and crest height, extending from the distal end to the bulbous head;
(c) a threaded tapered distal tip positioned at the distal end;
(d) a threaded proximal section having a cylindrical root, positioned distal to the bulbous head; and (e) a threaded distal section having a tapered root, positioned between the proximal section and the tapered distal tip, wherein the tapered root of the distal section gradually reduces in diameter from the proximal section to the distal tip, and wherein the crest diameter of the threads of the distal section narrows proportionally to the taper of the tapered root of the distal section.

2. The interference screw of claim 1, wherein the screw is made from a metal material.

3. The interference screw of claim 2, wherein the metal material is titanium or stainless steel.

4. The interference screw of claim 1, wherein the screw is cannulated.

5. The interference screw of claim 1, wherein the bulbous head has a recess in the head configured to receive a drive shaft.

6. The interference screw of claim 5, wherein the recess is a hexagonal socket

7. The interference screw of claim 1, wherein the threaded proximal section has a crest diameter of 9 mm, and wherein the crest diameter of the threaded distal section narrows from 9 mm to 7 mm (2 mm step).

8. The interference screw of claim 1, wherein the length of the threaded distal section is equal to or less than the length of the threaded proximal section

9. The interference screw of claim 1, wherein the overall length of the screw is 30 mm, the length of the threaded proximal section is 15 mm, and the length of the threaded distal section is 5 mm.

10. The interference screw of claim 9, wherein the length of the bulbous head is 5 mm and the length of the threaded distal tip is 5 mm.

11. The interference screw of claim 1, wherein the overall length of the screw is 25 mm, the length of the threaded proximal section is 10 mm, and the length of the threaded distal section is 5 mm.

12. The interference screw of claim 11, wherein the length of the bulbous head is 5 mm and the length of the threaded distal tip is 5 mm.

13. The interference screw of claim 1, wherein the overall length of the screw is 20 mm, the length of the threaded proximal section is 5 mm, and the length of the threaded distal section is 5 mm.

14. The interference screw of claim 13, wherein the length of the bulbous head is 5 mm and the length of the threaded distal tip is 5 mm.

15. A method of attaching a soft tissue graft to a bone mass for repair/replacement of damaged soft tissue, the method comprising:
(a) drilling a bone tunnel of a constant diameter into the bone mass to which the soft tissue graft is to be attached,
(b) placing the soft tissue graft partially or completely through the bone tunnel against a portion of the interior wall of the bone tunnel,
(c) positioning the distal end of the double lead interference screw of any one of claims 1 to 14 at the opening of the bone tunnel, and
(d) rotating the interference screw with sufficient torsional force while applying sufficient axial force to advance the screw into the bone tunnel until the proximal end of the screw is flush or substantially flush with the outer surface of the bone mass.

16. The method of claim 15, wherein the interference screw is cannulated.

17. The method of claim 16, wherein the method further comprises positioning a guide wire through the cannulated interference screw and into the bone tunnel in step 15 (c).

18. The method of claim 15, wherein the bulbous head of the interference screw has a recess in the head configured to receive a drive shaft.

19. The method of claim 18, wherein the method further comprises inserting the drive shaft of a screw driver into the recess in the head in step 15 (c), and wherein the torsional and axial forces in step 15 (d) are applied using the screw driver.

20. The method of claim 15, wherein the diameter of the bone tunnel is about 1 mm greater than crest diameter of the threaded proximal section of the interference screw.

21. The method of claim 15, wherein the soft tissue graft is a hamstring tendon or a quadriceps tendon.

22. The method of claim 15, wherein the bone mass is a femur bone and/or a tibia bone.

23. The method of claim 15, wherein the damaged soft tissue is an anterior cruciate ligament (ACL).
